(19) United States
(12) Patent Application Publication
(10) Pub. No.: US 2025/0017815 A1
(43) Pub. Date: Jan. 16, 2025

(54) WEARABLE DEVICE FOR TARGETED PERIPHERAL STIMULATION

(71) Applicant: Encora, Inc., Boston, MA (US)

(72) Inventors: Daniel Carballo, Boston, MA (US); Allison Davanzo, Green Cove Springs, FL (US)

(21) Appl. No.: 18/768,886

(22) Filed: Jul. 10, 2024

Related U.S. Application Data
(60) Provisional application No. 63/512,803, filed on Jul. 10, 2023.

(51) Int. Cl. A61H 23/02; G16H 10/60; G16H 20/30; G16H 50/20

(52) U.S. Cl. CPC A61H 23/02; G16H 10/60; G16H 20/30; G16H 50/20; A61H 2201/0157; A61H 2201/1207; A61H 2201/1638; A61H 2201/165; A61H 2201/501; A61H 2201/5058; A61H 2230/00

(57) ABSTRACT

In an aspect, a wearable device for targeted peripheral stimulation is presented. The wearable device includes a sensor configured to receive data and generate sensor output. The wearable device includes a processor in communication with the sensor. The wearable device includes a memory communicatively connected to the processor. The memory contains instructions configuring the processor to receive the sensor output from the sensor, the sensor output indicative of one or more movement disorder symptoms of a user. The processor is configured to determine a disease state of the one or more movement disorder symptoms. The processor is configured to generate a stimulation output based on the one or more movement disorder symptoms and the disease state of the movement disorder symptoms.

### CROSS REFERENCE TO RELATED APPLICATION

This application claims priority to U.S. Provisional Application No. 63/512,803, filed Jul. 10, 2023, the entirety of both of which is incorporated herein by reference.

### TECHNICAL FIELD

The present disclosure relates to wearable devices. In particular, the present disclosure relates to wearable devices for targeted peripheral stimulation.

### SUMMARY

In an aspect, a wearable device for targeted peripheral stimulation is presented. The wearable device includes a sensor configured to receive data and generate sensor output. The wearable device includes a processor in communication with the sensor. The wearable device includes a memory communicatively connected to the processor. The memory contains instructions configuring the processor to receive the sensor output from the sensor, the sensor output indicative of one or more movement disorder symptoms of a user. The processor is configured to determine a stage of an onset of the one or more movement disorder symptoms. The processor is configured to generate a stimulation output based on the one or more movement disorder symptoms and the stage of the onset of the movement disorder symptoms. The processor is configured to command a stimulator in communication with the processor to apply the stimulation output to a peripheral nervous system of the user to reduce the one or more movement disorder symptoms.

In another aspect, a method of targeted peripheral stimulation is presented. The method includes placing a wearable device on a user. The method includes detecting one or more movement disorder symptoms of the user through a sensor of the wearable device. The method includes calculating a stage of an onset of the one or more movement disorder symptoms. The method includes stimulating a peripheral nervous system of the user through a stimulator of the wearable device in response to the one or more movement disorder symptoms. The method includes detecting a responsiveness of the user through the sensor of the wearable device. The method includes stimulating the peripheral nervous system of the user based on the detected responsiveness and the stage of the onset of the one or more movement disorder symptoms.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures (or drawings). A brief introduction of the figures is below.

FIG. 1 is a block diagram of a wearable device for targeted peripheral stimulation;

FIG. 2 is a block diagram of a waveform parameter selection of a wearable device;

FIG. 3 is a block diagram of waveform parameter selection through a smartphone application in communication with a wearable device;

FIG. 4 is a block diagram of a feature extraction process that may be performed by a wearable device;

FIG. 5 illustrates a flowchart of a method of target peripheral stimulation;

FIG. 6 is a graph showing baseline Unified Parkinson's Disease Rating Scale (UPDRS) scores of subjects;

FIG. 7 is a graph showing median percent change and absolute change in symptoms of early onset Parkinson's disorder and late onset Parkinson's disorder subjects;

FIG. 8 is a graph showing results of baseline and stimulated EOPD subjects;

FIG. 9 is a graph showing results of baseline and stimulated LOPD subjects;

FIG. 10 is a graph illustrate in an improvement in BF-ADL scores of various ages of subjects;

FIG. 11 is an illustration of a wearable device; and

FIG. 12 is an exploded view of the wearable device of FIG. 11.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Aspects of the present disclosure may be used to provide for an interchangeability of one or more stimulators of wearable devices, such as wearable neurostimulation devices. In some embodiments, assemblies, systems, and methods herein may provide for increased durability of wearable neurostimulation devices, such as increase in mechanical stress, moisture, temperature changes, repeated electrical or vibratory stimulation, and/or other parameters. Embodiments of the present disclosure may be used to provide for a low-profile electrical connector system that may enable long-lasting electrical connections between a stimulation assembly and/or a control/power supply assembly.

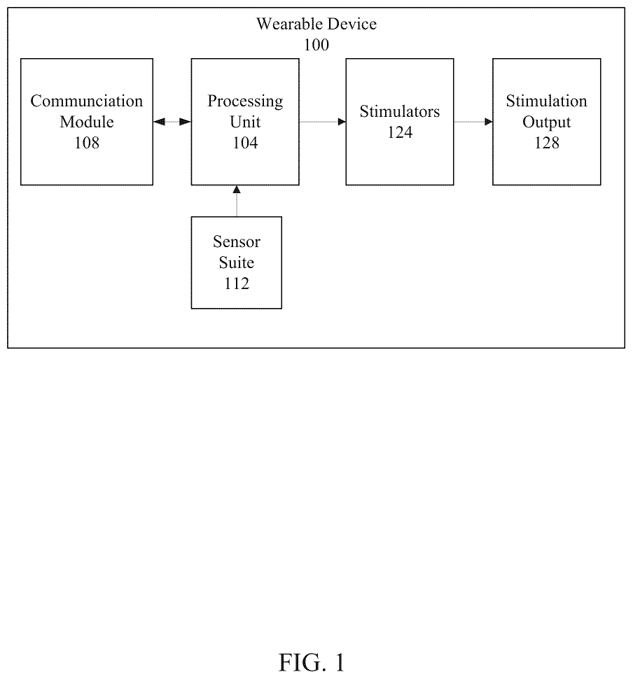

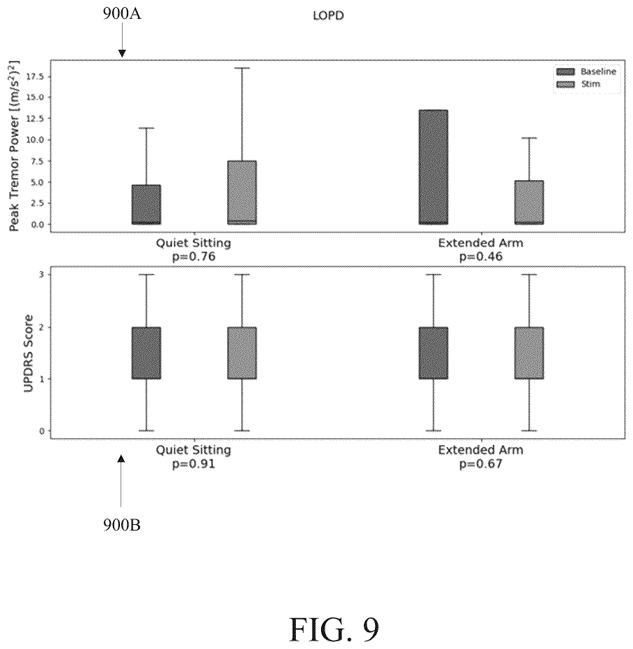

FIG. 1 illustrates a system 100 for targeted peripheral stimulation in accordance with an embodiment of the present invention. The system 100 may include a wearable device 100. The wearable device 100 may include a processor, such as processing unit 104, and a memory communicatively connected to the processing unit 104. A memory of the wearable device 100 may contain instructions configuring the processing unit 104 of the wearable device 100 to perform various tasks. The wearable device 100 may include a communication module 108. A “communication module” as used throughout this disclosure is any form of software and/or hardware capable of transmission of electromagnetic energy. For instance, the communication module 108 may be configured to transmit and receive radio signals, Wi-Fi signals, Bluetooth® signals, cellular signals, and the like. The communication module 108 may include a transmitter, receiver, and/or other component. A transmitter of the communication module 108 may include, but is not limited to, an antennae. Antennas of the communication module 108 may include, without limitation, dipole, monopole, array, loop, and/or other antenna types. A receiver of the communication module 108 may include an antenna, such as described previously, without limitation. The communication module 108 may be in communication with the processing unit 104. For instance, the processing unit 104 may be physically connected to the communication module 108 through one or more wires, circuits, and the like. The processing unit 104 may command the communication module 108 to send and/or receive data transmissions to one or more other devices. For instance, and without limitation, the communication module 108 may transmit vibrational stimulus data, motion data of the user's body 150, electrical activity of the user's muscles 164, and the like. In some embodiments, the communication module 108 may transmit treatment data. Treatment data may include, without limitation, symptom severity, symptom type, vibrational stimulus 13 frequency, data from the sensor suite 112, and the like. The communication module 108 may communicate with one or more external computing devices such as, but not limited to, smartphones, tablets, laptops, desktops, servers, cloud-computing devices, and the like. The wearable device 100 may be as described further below with reference to FIG. 9.

With continued reference to FIG. 1, the wearable device 100 may include one or more sensors. A “sensor” as used throughout this disclosure is an element capable of detecting a physical property. Physical properties may include, but are not limited to, kinetics, electricity, magnetism, radiation, thermal energy, and the like. In some embodiments, the wearable device 100 may include a sensor suite 112. A “sensor suite” as used throughout this disclosure is a combination of two or more sensors. The sensor suite 112 may have a plurality of sensors, such as, but not limited to, two or more sensors. The sensor suite 112 may have two or more of a same sensor type. In other embodiments, the sensor suite 112 may have two or more differing sensor types. For instance, the sensor suite 112 may include an electromyography sensor (EMG) and an inertial measurement unit (IMU). An IMU may be configured to detect and/or measure a body's specific force, angular rate, and/or orientation. Other sensors the sensor suite 112 may include are accelerometers, gyroscopes, impedance sensors, temperature sensors, and/or other sensor types, without limitation. The sensor suite 112 may be in communication with the processing unit 104. A communication between the sensor suite 112 and the processing unit 104 may be an electrical connection in which data may be shared between the sensor suite 112 and the processing unit 104. In some embodiments, the sensor suite 112 may be wirelessly connected to the processing unit 104, such as through, but not limited to, a Wi-Fi, Bluetooth®, or other connection. In some embodiments, the wearable device 100 may be the same as the wearable device described in U.S. application Ser. No. 16/563,087, filed Sep. 6, 2019, and titled “Apparatus and Method for Reduction of Neurological Movement Disorder Symptoms Using Wearable Device”, the entirety of which is incorporated herein by reference.

One or more sensors of the sensor suite 112 may be configured to receive data from a user, such as from a user's body. Data received by one or more sensors of the sensor suite 112 may include, but is not limited to, motion data, electric data, and the like. Motion data may include, but is not limited to, acceleration, velocity, angular velocity, and/or other types of kinetics. In some embodiments an IMU of the sensor suite 112 may be configured to receive motion from a user's body. Motion may include, without limitation, vibration, acceleration, muscle contraction, and/or other aspects of motion. Motion may be generated from one or more muscles of a user's body. Muscle may include, but are not limited to, wrist muscles, hand muscles, forearm muscles, and/or other muscles. In an embodiment, motion generated from one or more muscles of a user's body may be involuntarily generated by one or more symptoms of a movement disorder of the user's body. A movement disorder may include, without limitation, Parkinson's disease (PD), post stroke recovery, and the like. Symptoms of a movement disorder may include, but are not limited to, stiffness, freezing of gait, tremors, shaking, involuntary muscle contraction, and/or other symptoms. In other embodiments, motion generated from muscles of a user's body may be voluntary. For instance, a user may actively control one or more of their muscles, which may generate motion that may be detected and/or received by a sensor of the sensor suite 112.

Still referring to FIG. 1, one or more sensors of the sensor suite 112 may be configured to receive electrical data, such as the electrical activity that may be generated by one or more of muscles of a user. Electric data may include, but is not limited to, voltages, impedances, currents, resistances, reactance values, waveforms, and the like. For instance, electrical activity may include an increase in current and/or voltage of one or more of muscles during a contraction of the one or more of the muscles. An EMG of the sensor suite 112 may be configured to receive and/or detect electrical activity generated by one or more muscles of a user. In some embodiments, one or more sensors of the sensor suite 112 may configured to generate sensor output. “Sensor output” as used in this disclosure is information generated by one or more sensing devices. Sensor output may include, but is not limited to, voltages, currents, accelerations, velocities, and/or other output. Sensor output generated from one or more sensors of the sensor suite 112 may be communicated to the processing unit 104, such as through a wired, wireless, or other connection. The processing unit 104 may be configured to determine a symptom of a movement disorder based on sensor output received from one or more sensors. The processing unit 104 may be configured to determine symptoms such as, but not limited to, stiffness, tremors, freezing of gait, and the like. Freezing of gait refers to a symptom of Parkinson's disease in which a person with Parkinson's experiences sudden, temporary episodes of inability to step forward despite an intention to walk. An abnormal gait pattern can range from merely inconvenient to potentially dangerous, as it may increase the risk of falls. Stiffness may refer to a muscle of a person with Parkinson's disease that may contract and become rigid without the person wanting it to. The processing unit 104 may compare one or more values of sensor output from the sensor suite 112 to one or more values associated with one or more symptoms of a movement disorder. For instance, the processing unit 104 may compare sensor output of one or more sensors of the sensor suite 112 to one or more stored values that may already be associated with one or more symptoms of a movement disorder. As a non-limiting example, acceleration of a user's arm of about 1 in/s to about 3 in/s may correspond to a symptom of a light tremor.

In some embodiments, the processing unit 104 may utilize a classifier or other machine learning model that may categorize sensor output to categories of symptoms of a movement disorder. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Processor 104 and/or another device may generate a classifier using a classification algorithm, defined as a processes whereby a processor derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, Fisher's linear discriminant, quadratic classifiers, decision trees, boosted trees, random forest classifiers, kernel estimation, learning vector quantization, and/or neural network-based classifiers.

With continued reference to FIG. 1, a classifier may be generated, as a non-limiting example, using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table.

The processor 104 may calculate a likelihood table by calculating probabilities of different data entries and classification labels. The processor 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, a classifier may be generated using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample. this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

A classifier may be trained with training data correlating motion data and/or electric data to symptoms of a movement disorder. Training data may be received through user input, external computing devices, and/or previous iterations of training. As a non-limiting example, an IMU of sensor suite 112 may receive motion data generated by one or more muscles of a user and may generate sensor output including acceleration values which may be communicated to the processing unit 104. The processing unit 104 may classify and/or categorize the sensor output to a symptom of freezing of gait.

Still referring to FIG. 1, the processing unit 104 may train a classifier with training data correlating motion and/or electrical data to symptoms of a movement disorder. In other embodiments, training of a classifier and/or other machine learning model may occur remote from the processor 104 and the processor 104 may be sent one or more trained models, weights, and the like of a classifier, machine learning model, and the like. Training data may be received by user input, through one or more external computing devices, and/or through previous iterations of processing. A classifier may be configured to input sensor output, such as output of the sensor suite 112, and categorize the output to one or more groups, such as, but not limited to, tremors, stiffness, freezing of gait, and the like.

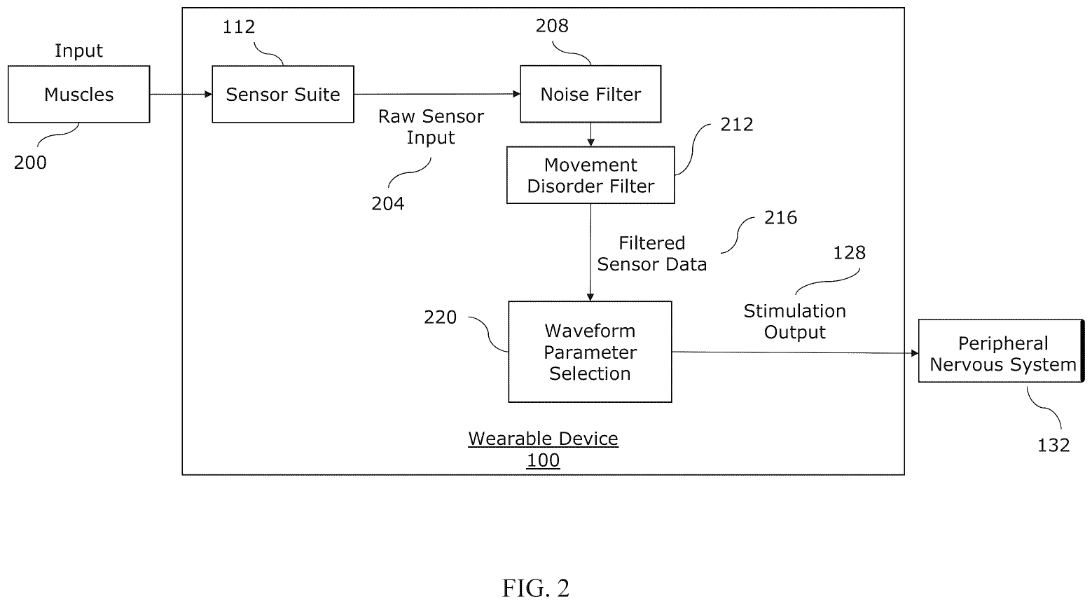

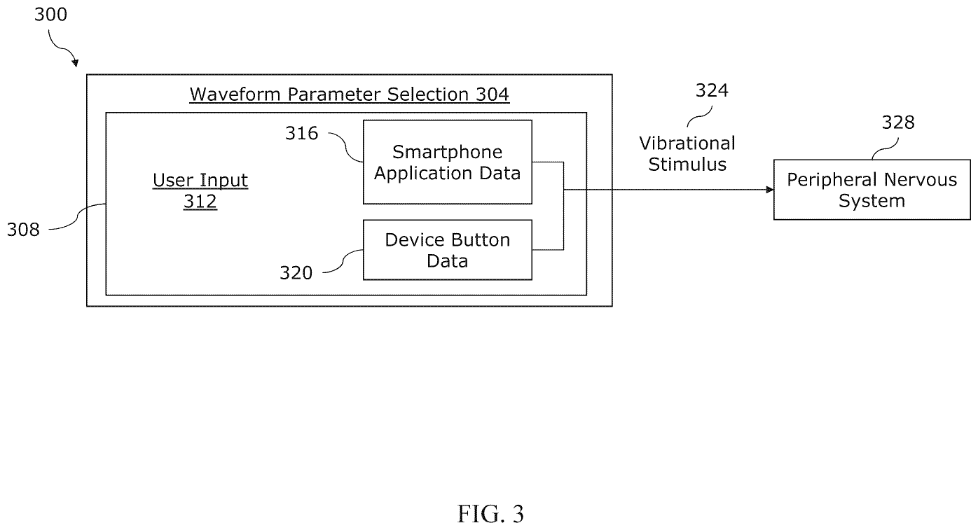

The processing unit 104 may calculate a stimulation output 128 based on sensor output generated by one or more sensors of the wearable device 100. A “stimulation output” as used in this disclosure is a signal having a frequency. Stimulation output 128 may be generated as a vibrational, electrical, audial, and/or other output. Stimulation output 128 may include one or more waveform outputs. A waveform output may include one or more parameters such as frequency, phase, amplitude, channel index, and the like. A channel index may include a channel of mechanoreceptors and/or of an actuator to be used. For instance, a channel index may include one or more channels of mechanoreceptors, actuators to stimulate the mechanoreceptors, and/or a combination thereof. The processing unit 104 may select one or more parameters of a waveform output based on received sensor output from one or more sensors of the sensor suite 112. In other embodiments, waveform parameters of stimulation output 128 may be selected by the user. As a non-limiting example, a user may select stimulation and/or waveform parameters of stimulation output 128 from a predefined list of waveforms using buttons or other interactive elements on the wearable device 100. A predefined list of stimulation outputs 128 may include one or more waveforms having various frequencies, amplitudes, and the like, without limitation. A predefined list of stimulation outputs 128 may be generated through previous iterations of stimulation output 128 generation. In other embodiments, a predefined list of stimulation outputs 128 may be entered by one or more users. In some embodiments, a predefined list of stimulation outputs 128 may include stimulation outputs 128 for specific symptoms, such as, but not limited to, freezing of gait, tremors, stiffness, and the like. In some embodiments, a user may select specific waveform parameters using an external computing device such as, but not limited to, a smartphone, laptop, tablet, desktop, smartwatch, and the like, which may be in communication with the processing unit 104 through the communication module 108. Stimulation output 128 generation may be described in further detail below with reference to FIGS. 2-3.

In some embodiments, the processing unit 104 may communicate stimulation output 128 with one or more stimulators 124 of the wearable device 100. A “stimulator” as used in this disclosure is any device capable of producing a stimulating output. For instance, stimulators 124 may include electric, mechanical, thermal, audial, and/or other types of stimulators 124. The wearable device 100 may include one or more stimulators 124. For instance, the wearable device 100 may include two or more stimulators 124. In some embodiments, the wearable device 100 may include two or more stimulators 124 of differing types, such as a mechanical stimulator and an electrical stimulator, an electrical stimulator and an audial stimulator, and the like. Stimulators 124 of the wearable device 100 may be positioned to provide stimulus, such as through stimulation output 128, to specific parts of a user's body. For instance, the wearable device 100 may include one or more stimulators 124 that may be positioned to stimulate one or more portions of a user's peripheral nervous system. A “peripheral nervous system” as used in this disclosure refers to the part of nervous system that is outside the central nervous system (CNS). The peripheral nervous system may include one or more nerves and/or tissues. The peripheral nervous system may include one or more ganglion. “Ganglion” refers to a group of neuron cells bodies in the peripheral nervous system. Ganglion may include dorsal root ganglia and/or trigeminal ganglia. Nerves and/or tissues of a peripheral nervous system may be referred to as “peripheral nerves” and “peripheral tissues,” respectively. The processing unit 104 may be configured to target peripheral nerves and/or tissues of a peripheral nervous system of a user. Peripheral nerves and/or tissues may be located in a user's wrist, arm, neck, and/or other parts of a user. In some embodiments, stimulation output 128 may be delivered to one or more peripheral nerves and/or tissues of a user. In some embodiments, stimulation output 128 may be provided to one or more mechanoreceptors of a user's body. Mechanoreceptors” as used throughout this disclosure refer to cells of a human body that respond to mechanical stimuli. Mechanoreceptors may include proprioceptors and/or somatosensors. Proprioceptors may include head stems of muscles innervated by the trigeminal nerve. Proprioceptors may be part of one or more areas of a user's limbs, such as, but not limited to, wrists, hands, legs, feet, arms, and the like. Somatosensors 160 may include cells having receptor neurons located in the dorsal root ganglion.

In some embodiments, the stimulators 124 may be positioned along a wristband of the wearable device 100. The wearable device 100 may include, in an embodiment, four or more stimulators 124 that may be equidistant from one another and positioned within a wristband of the wearable device 100. In other embodiments, the stimulators 124 may be positioned on a surface of a housing of the wearable device 100. The stimulators 124 may include, but are not limited to, piezoelectric motors, electromagnet motors, linear resonant actuators (LRA), eccentric rotating mass motors (ERMs), and the like. The stimulators 124 may be configured to vibrate at up to or more than 200 kHz, in an embodiment. The stimulators 124 may draw energy from one or more batteries from the wearable device 100. For instance, the stimulators 124 may draw about 5 W of power from a battery of the wearable device 100. In some embodiments, stimulators 124 may have a max current draw of about 90 mA, a current draw of about 68 mA, a 34 mA current draw at 50% duty cycle, and may have a voltage of about 0V to about 5V, without limitation. In some embodiments, a suprasensory vibration may have an acceleration greater than or equal to 50 mGᵣₘₛ. In some embodiments, a suprasensory vibration produced by one or more stimulators 124 may have an acceleration between 180 mGᵣₘₛ and 1.8 Grms. In some embodiments, a subsensory vibration produced by one or more stimulators 124 may have an acceleration between 0 and 50 mGᵣₘₛ.

Still referring to FIG. 1, the processing unit 104 may be configured to command the stimulators 124 to apply the stimulation output 128 to one or more mechanoreceptors of the users body. The stimulation output 128 may include a waveform output calculated by the processing unit 104 and may be applied to the user's body through the stimulators 124. The stimulation output 128 may be applied to the mechanoreceptors or other peripheral nerves and/or tissues, which may cause the mechanoreceptors to generate one or more afferent signals. An “afferent signal” as used in this disclosure is a neuronal signal in a form of action potentials that are carried toward target neurons. Afferent signals may be communicated to the peripheral nervous system (PNS) of a user's body. A user's brain may communicate efferent signals to the PNS 172 through the spinal cord. “Efferent signals” as used in this disclosure are signals that carry motor information for a muscle to take an action. Efferent signals may include one or more electrical signals that may cause one or more muscles to contract or otherwise move. For instance, the PNS may input afferent signals and communicate the afferent signals to the brain through the spinal cord. The brain may generate one or more efferent signals and communicate the efferent signals to the PNS through the spinal cord. The PNS may communicate the efferent signals to the muscles.

With continued reference to FIG. 1, the processing unit 104 may act in a closed-loop system. For instance the processing unit 104 may act in a feedback loop between the data generated from one or more muscles of a user and the stimulation output 128 generated by the stimulators 124. Further, a closed-loop system may extend through and/or to the PNS, central nervous system (CNS), brain, and the like of a user's body based on afferent signals and efferent signals. In some embodiments, the processing unit 104 may be configured to act in one or more modes. For instance, the processing unit 104 may act in a first and a second mode. A first mode may include monitoring movements of a user's body passively to detect one or more movement disorder symptoms above a threshold. A threshold may include a root mean squared acceleration of 100 mG or 500 mGA, in an embodiment. A threshold may be set by a user and/or determined through the processing unit 104 based on historical data. Historical data may include sensor and/or stimulation output 128 data of a user over a period of time, such as, but not limited to, minutes, hours, weeks, months, years, and the like. A threshold may include, without limitation, one or more acceleration, pressure, current, and/or voltage values. In some embodiments, upon a threshold being reached, the processing unit 104 may be configured to act in a second mode in which the processing unit 104 commands the stimulators 124 to provide the stimulation output 128 to one or more peripheral nerves and/or tissues of a user.

In some embodiments, the processing unit 104 may utilize a stimulation selection algorithm. A stimulation selection algorithm may input current stimulation parameters of stimulation output 128 and/or extracted features of sensor data and through a model free policy optimization algorithm may generate new stimulation parameters. Model free policy optimization may include, but is not limited to, Argmin, Q-learning, neural networks, genetic algorithms, differential dynamic programming, iterative quadratic regulator, and/or guided policy search. The processing unit 104 may continuously update stimulation parameters of stimulation output 128 utilizing a stimulation selection algorithm. For instance, new stimulation parameters may become current stimulation parameters in a subsequent cycle, and the processing unit 104 may repeat the stimulation selection algorithm.

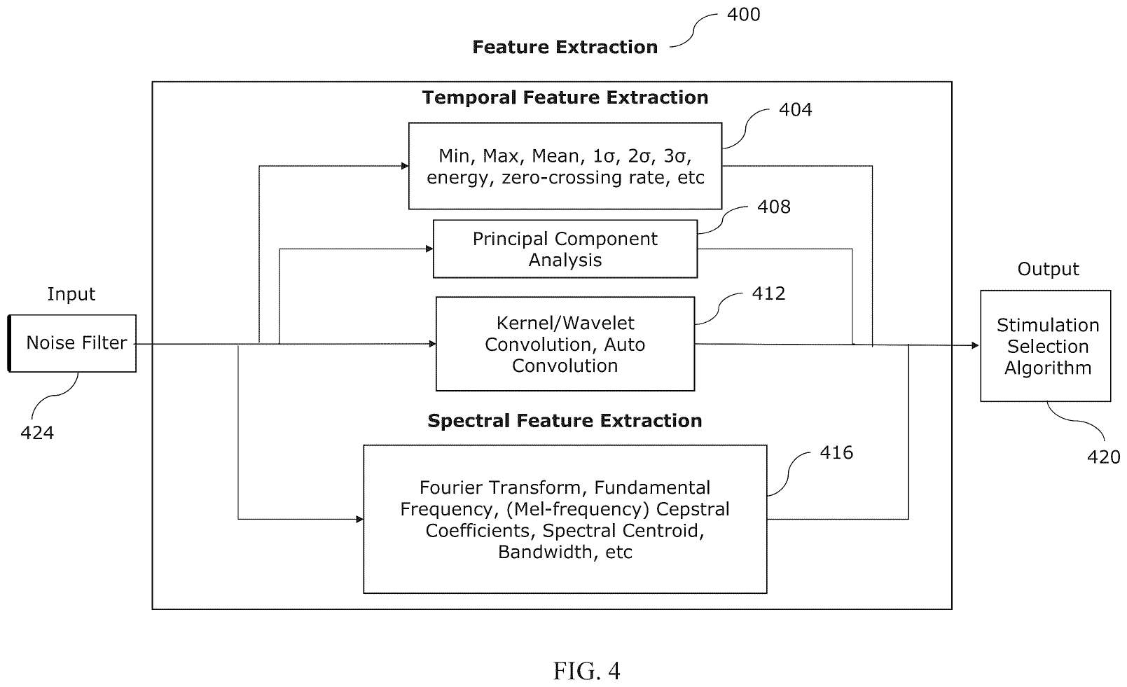

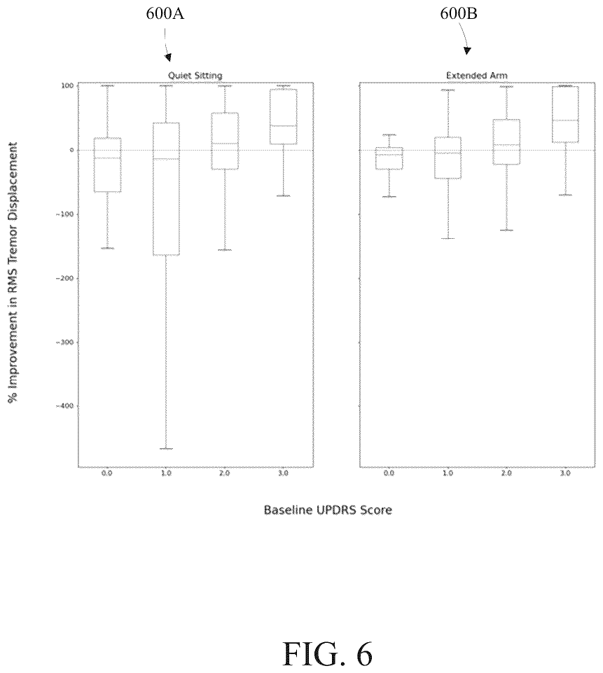

In some embodiments, the processing unit 104 may operate in a third mode. A third mode may include a diagnostic mode. A diagnostic mode may be a mode in which the processing unit 104 is configured to detect a responsiveness of a user to stimulation therapy, such as stimulation output 128. For instance, a responsiveness of a user may be determined through sensor data generated by the sensor suite 112. Sensor data may be generated from a user's body response to stimulation output 128. For instance, a severity of one or more movement disorder symptoms of a user may be reduced in response to receiving stimulation output 128. In some embodiments, the processing unit 104 may be configured to calculate a severity level of one or more movement disorder symptoms of a patient. A “severity level” as used in this disclosure refers to a classification of intensity of one or more movement disorder symptoms. For instance and without limitation, a severity level may include, but is not limited to, low, moderate, high, or severe. A severity level may be determined by an amplitude, frequency, and/or other parameters of one or more movement disorder symptoms. For instance and without limitation, one or more movement disorder symptoms may include a tremor, which may have an amplitude. A normal to moderate severity level may include about less than 1 cm in tremor amplitude. A mild to sever severity level may include about 1 cm or greater than about 1 cm in tremor amplitude. A high severity level may include about 2 cm or greater than about 2 cm in tremor amplitude. A severity level may be determined according to a baseline tremor score, as described in further detail below with reference to FIGS. 6-9. Severity levels may include any type of movement disorder symptoms as described throughout this disclosure, without limitation. A severity may be reduced by about 10%, 20%, 30%, 40%, 50%, 60%, 70%, 80%, or more than 80%. In some embodiments, a severity reduction may be correlated by the processing unit 104 to determine a level of responsiveness of a user to stimulation therapy. A level of responsiveness may include, but is not limited to, low responsiveness, low to average responsiveness, average responsiveness, average to high responsiveness, or high responsiveness. For instance and without limitation, a reduction in severity of one or more movement disorder symptoms by about 10% to about 30% may be classified as low responsiveness, a reduction in severity of one or more movement disorder symptoms by about 30% to about 60% may be classified as average responsiveness, and a reduction in severity of one or more movement disorder symptoms by about 60% or more may be classified as a high responsiveness. In some embodiments, an average responsiveness may include a reduction in severity of one or more movement disorder symptoms by lower than about 30% to about 60%, such as, but not limited to, about 10% to about 20%, about 10% to about 30%, about 10% to about 40%, or any ranges in between these ranges. A low responsiveness may include a reduction in severity of one or more movement disorder symptoms by about 5% to about 10%, about 5% to about 15%, about 5% to about 20%, or any ranges within these ranges, without limitation. In some embodiments, a high responsiveness may include a reduction in severity by one or more movement disorder symptoms by greater than about 70%. In some embodiments, responsiveness may be correlated to a stage of an onset of one or more movement disorder symptoms. For instance, users with a stage of early onset of one or more movement disorder symptoms may be higher responders than users with a stage of late onset of one or more movement disorder symptoms. In some embodiments, responsiveness may be correlated to patient data and/or demographic data. Patient data, such as demographic data, may include, but is not limited to, age, sex, weight, height, muscle mass, skeletal muscle mass, body fat percentage, age of an initial onset of one or more movement disorder symptoms, and/or other data. In some embodiments, patients with a younger age may have a higher responsiveness to stimulation compared to patients with an older age, while patients with an older age may have a lower responsiveness to stimulation compared to patients with a younger age. In some embodiments, responsiveness may be correlated to a period of time since an onset of one or more movement disorder symptoms. For instance and without limitation, a smaller period of time between an initial onset of one or more movement disorder symptoms may correlate to a higher responsiveness to stimulation in a user, while a longer period of time between an initial onset of one or more movement disorder symptoms and stimulation may correlate to a lower responsiveness to stimulation in a user. Responsiveness may correlate based at least in part on a current age of a user and/or an initial age of the user at an onset of one or more movement disorder symptoms. Differences in responsiveness may be as shown below with reference to FIGS. 4-7.

Referring still to FIG. 1, In some embodiments, the processing unit 104 may be configured to determine a disease state of a user. A “disease state” as used in this disclosure refers to an overall progress and/or severity of one or more movement disorder symptoms of an individual. For instance and without limitation, a disease state may include a stage of an onset of one or more movement disorder symptoms, a severity level of one or more movement disorder symptoms, and/or a stage of one or more movement disorder symptoms. In some embodiments, processing unit 104 may be configured to determine a disease state of a user based on sensor data generated locally and/or from sensor data received via one or more external computing devices. In some embodiments, processing unit 104 may be configured to determine a disease state of a user based on motion and/or electrical activity of a user or other data that may be measured by sensor suite 112. For instance, the processing unit 104 may determine an disease state including an early stage, intermediate stage, or late stage of an onset of one or more movement disorder symptoms or any range in between these stages. In some embodiments, the processing unit 104 may determine a disease state locally. In other embodiments, the processing unit 104 may communicate sensor data to an external computing device, such as but not limited to a laptop, server, desktop, smartphone, or other device. An external computing device may calculate a disease state of a and may communicate a calculated stage to the processing unit 104, such as through communication module 108. The processing unit 104 may adjust one or more parameters of stimulation output 128 based on a calculated disease state of a user. For instance and without limitation, a higher amplitude and/or frequency of a waveform of stimulation output 128 may be used for users with a disease state including a late stage onset of one or more movement disorder symptoms, which may be due to their lower responsiveness to stimulation output 128. A lower amplitude and/or frequency of a waveform of stimulation output 128 may be used for users with a disease state including an early stage onset of one or more movement disorder symptoms, which may be due to their higher responsiveness to stimulation output 128. In some embodiments, a machine learning model may be used to calculate adjusted parameters of stimulation output 128. For instance, a machine learning model may be trained with training data correlating one or more parameters of a disease state with one or more parameters of stimulation output 128. Training data may be received via user input, external computing devices, and/or previous iterations of processing. In some embodiments, a classifier may be used to classify sensor data generated by the sensor suite 112 to various stages of disease states. A classifier may be trained with training data correlating sensor data, such as movement and electrical activity, with disease states. Training data may be received via user input, external computing devices, and/or previous iterations of processing. The processing unit 104 may operate one or more machine learning models and/or classifiers locally. In other embodiments, the processing unit 104 may be in communication with one or more external computing devices via communication module 108. External computing devices 108 may be operable to run one or more machine learning models and/or classifiers. In some embodiments, parameters of one or more machine learning models or classifiers may be sent to the processing unit 104 from one or more external computing devices via the communication module 108. The processing unit 104 may utilize one or more parameters of a machine learning model and/or classifier to run a local version of a machine learning model and/or classifier.

Referring now to FIG. 2, a block diagram of a waveform parameter selection system 220 is presented. The system 220 may be local to the wearable device 100, such as by being processed by the processor 104. In other embodiments, the system 220 may be ran through an external computing device, such as, but not limited to, smartphones, tablets, desktops, laptops, servers, cloud-computing devices, and the like. The processing unit 104 may be configured to process raw sensor input 604 received from the sensor suite 112 based on activity of one or more muscles 200 of a user. The raw sensor input 204 may include unprocessed and/or unfiltered sensor data gathered and/or generated by the sensor suite 112. In some embodiments, the processing unit 104 may place the raw sensor input 204 through one or more filters. Filters may include, but are not limited to, noise filters. Filters may include non-linear, linear, time-variant, time-invariant, casual, non-casual, discrete-time, continuous-time, passive, active, infinite impulse response (IIR), finite impulse response (FIR), and the like. The processing unit 104 may use one or more filters to remove noise from the sensor output, such as the noise filter 208. Noise may include unwanted modifications to a signal, such as unrelated sensor output of one or more sensors of the sensor suite 112. The noise filter 208 may use either knowledge of the output waveform to subtract from the sensed waveform or knowledge of the timing of the output waveform to limit sensing to the “off” phases of a pulsing stimulation. In some embodiments, the processing unit 104 may use a filter to remove all information unrelated to a movement disorder, such as through the movement disorder filter 212. Information unrelated to a movement disorder may include specific frequencies and/or ranges of frequencies that may be outside of an indication of a movement disorder. As a non-limiting example, a tremor may have a frequency of about 3 Hz to about 15 Hz, and any frequencies outside of this range may be unrelated to the tremor and subsequently removed through one or more filters. As another non-limiting example, classical rest tremor, isolated postural tremor, and kinetic tremor during slow movement may be about 3 Hz to about 7 Hz, 4 Hz to about 9 Hz, and 7 Hz to about 12 Hz, respectively. The processing unit 104 may be configured to filter any frequencies outside of any of the ranges described above. In some embodiments, the processing unit 104 may be configured to extract a fundamental tremor frequency through spectral analysis. A fundamental tremor frequency may be used in one or more filters as a digital bandpass filter with a cutoff frequencies about and below the fundamental frequency. The processing unit 104 may be configured to implement and/or generate one or more filters based on a patient's specific fundamental tremor frequency. The movement disorder filter 212 may be any filter type. In some embodiments, the movement disorder filter 212 may include a 0-15 Hz bandpass filter configured to eliminate any other signal components not caused by a movement disorder. In other embodiments, the movement disorder filter 212 may include a bandpass filter with an upper limit greater than 15 Hz, without limitation. The processing unit 104 may use the movement disorder filter 212 to determine extraneous movement of a user by removing noise unrelated to an extraneous movement of the user. The processing unit 104 may utilize three or more filters, in an embodiment. The processing unit 104 may first use the noise filter 208 to remove noise from the raw sensor input 204 and subsequently use a second filter, such as the movement disorder filter 212, to remove all information unrelated to a movement disorder. In some embodiments, after processing sensor output through one or more filters, filtered sensor data 216 may be generated. In some embodiments, one or more features may be extracted from the filtered sensor data 216. Extraction may include retrieving temporal, spectral, or other features of the filtered sensor data 216. Temporal features may include, but are not limited to, minimum value, the maximum value, first three standard deviation values, signal energy, root mean squared (RMS) amplitude, zero crossing rate, principal component analysis (PCA), kernel or wavelet convolution, or auto-convolution. Spectral features may include, but are not limited to, the Fourier Transform, fundamental frequency, (Mel-frequency) Cepstral coefficients, the spectral centroid, and bandwidth. The processing unit 104 may input the filtered sensor data 216 and/or extraction features of the filtered sensor data 616 into the waveform parameter algorithm 220.

The waveform parameter selection algorithm 220 may be a parameter selection algorithm. A parameter selection algorithm may include an algorithm that determines one or more parameters of an output. The waveform parameter selection 220 may include, without limitation, a classification algorithm such as a logistic regression, naïve bayes, decision tree, support vector machine, neural network, random forest, and/or other algorithm. In some embodiments, the waveform parameter selection 220 may be an argmax (FFT) algorithm. The waveform parameter selection 220 may include a calculation of a mean, median, interquartile range, Xth percentile signal frequency, root mean square amplitude, power, log (power), and/or linear or non-linear combination thereof. For instance, and without limitation, the waveform parameter selection 220 may modify a frequency, amplitude, peak-to-peak value, and the like of one or more waveforms. The waveform parameter algorithm 220 that may modify one or more parameters of a waveform output applied to the peripheral nervous system 232, such as the stimulation output 128. In some embodiments, the waveform parameter algorithm 220 may be configured and/or programmed to determine a set of waveform parameters based on a current set of waveform parameters and/or the filtered sensor data 216. As a non-limiting example, the filtered sensor data 216 may include an amplitude of a tremor. The waveform parameter algorithm 220 may compare the tremor amplitude with a current set of waveform parameters to a tremor amplitude observed with a previous set of waveform parameters to determine which of the two sets of waveform parameters results in a lowest tremor amplitude. The set with a lowest resulting tremor amplitude may be used as a baseline for a next iteration of the waveform parameter selection 220, which may compare this baseline to a new set of waveform parameters. The waveform parameter selection 220 may utilize one or more of a Q-learning model, one or more neural networks, genetic algorithms, differential dynamic programming, iterative quadratic regulator, and/or guided policy search. The waveform parameter selection 220 may determine one or more new waveform parameters from a current set of applied waveform parameters based on an optimization model to best minimize a symptom severity of a user. An optimization model may include, but is not limited to, discrete optimization, continuous optimization, and the like. For instance, the waveform parameter selection 220 may utilize an optimization model that may be configured to input the filtered sensor data 216 and/or current waveform parameters of the vibrational stimulus 13 and output a new selection of waveform parameters that may minimize symptom severity of a user. Symptom severity may include, but is not limited to, freezing of gait, stiffness, tremors, and the like.

In some embodiments, the stimulation output 128 may target afferent nerves chosen from the set consisting of the somatosensory cutaneous afferents of the C5-T1 dermatomes and the proprioceptive afferents of the muscles and tendons of the wrist, fingers, and thumb, without limitation. In an embodiment, the stimulation output 128 may be applied around a circumference of a user's wrist which may allow for stimulation of five distinct somatosensory channels via the C5-T1 dermatomes as well as an additional fifteen proprioceptive channels via the tendons passing through the wrist, which may allow for a total of twenty distinct channels. The waveform parameter selection 220 may be configured to generate one or more waveform parameters specific to one or more proprioceptive and/or somatosensory channels. For instance, and without limitation, the waveform parameter selection 220 may select a single proprioceptive channel through a C5 dermatome to apply the stimulation output 128 to. In another instance, and without limitation, the waveform parameter selection 220 may select a combination of a C5 dermatome and T1 dermatome channel. In some embodiments, the waveform parameter selection 620 may be configured to generate a multichannel waveform by generating one or more waveform parameters for one or more proprioceptive and/or somatosensory channels. Channels of a multichannel waveform may be specific to one or more proprioceptive and/or somatosensory channels. In some embodiments, each transducer of a plurality of transducers may each generate a waveform output for a specific proprioceptive and/or somatosensory channel, where each channel may differ from one another, be the same, or a combination thereof. The waveform parameter selection 220 may select any combination of proprioceptive and/or somatosensory channels, without limitation. The waveform parameter selection 220 may select one or more proprioceptive channels to target based on one or more symptoms of a movement disorder. For instance and without limitation, the waveform parameter selection 220 may select both a T1 and C5 channel for stimulation based on a symptom of muscle stiffness. In some embodiments, the waveform parameter selection 220 may include a stimulation machine learning model. A stimulation machine learning model may include any machine learning model as described throughout this disclosure, without limitation. In some embodiments, a stimulation machine learning model may be trained with training data correlating sensor data and/or waveform parameters to optimal waveform parameters. Training data may be received through user input, external computing devices, and/or previous iterations of processing. A stimulation machine learning model may be configured to input the filtered sensor data 216 and/or a current set of waveform parameters and output a new set of waveform parameters. A stimulation machine learning model may be configured to output specific targets for vibrational stimulus, such as one or more proprioceptive and/or somatosensory channels as described above, without limitation. As a non-limiting example, a stimulation machine learning model may input the filtered sensor data 216 and output a set of waveform parameters specific to a C6 and C8 proprioceptive channel. The stimulation output 128 may be applied to one or more mechanoreceptors. In some embodiments, where this process happens externally to the wearable device 100, a computing device may communicate one or more waveform parameters to the wearable device 100.

The waveform parameter selection 220 may generate a train of waveform outputs. A train of waveform outputs may include two or more waveform outputs that may be applied to a user sequentially. Periods of time between two or more waveform outputs of a train of waveform outputs may be, without limitation, milliseconds, seconds, minutes, and the like. Each waveform output of a train of waveform outputs may have varying parameters, such as, but not limited to, amplitudes, frequencies, peak-to-peak values, and the like. In some embodiments, a train of waveform outputs may include a plurality of waveform outputs with each waveform output having a higher frequency than a previous waveform output. In some embodiments, each waveform output may have a lower or same frequency than a previous waveform output. The waveform parameter selection 220 may provide a train of waveform outputs until a waveform output reaches a frequency that results in a suppressed output of extraneous movement of a user.

Still referring to FIG. 2, the wearable device 100 may be configured to act in one or more settings. Settings of the wearable device 100 may include one or more modes of operation. A user may be configured to select one or more settings of the wearable device 100 through interactive elements, such as buttons, touch screens, and the like, and/or through a remote computing device, such a through an application, without imitation. Interactive elements and applications may be described in further detail below with reference to FIG. 3.

Settings of the wearable device 100 may include an automatic setting, a tremor reduction setting, a freezing of gait setting, a stiffness setting, and/or an adaptive mode setting. An automatic setting of the wearable device 100 may include the processing unit 104 automatically selecting a best waveform output based on data generated from one or more sensors of the sensor suite 112. For instance, the waveform parameter selection 220 may select one or more waveform parameters that are generally best suited for current sensor data, such as filtered sensor data 216. An automatic mode of the wearable device 100 may be based on a plurality of data generated from a plurality of users using the wearable device 100 to find one or more averages, standard deviations, and the like, of stimulation output 128. In some embodiments, generating an automatic mode of the wearable device 100 may include crowd-sourcing from one or more users. A cloud-computing system may be implemented to gather data of one or more users.

Still referring to FIG. 2, the wearable device 100 may be configured to act in a tremor reduction setting. A tremor reduction setting may include the waveform parameter selection 220 giving more weight or value to filtered sensor data 216 that corresponds to tremors, while lessening weights or values of other symptoms. The waveform parameter selection 220 may be configured to generate one or more waveform parameters that optimize a tremor reduction of a tremor of a user. Optimizing a tremor reduction of a user may include minimizing weights, values, and/or waveform parameters for other symptoms, such as freezing of gait, stiffness, and the like. Likewise, a freezing of gait setting may optimize a reduction in a freezing of gait of a user, a stiffness setting may optimize a reduction in stiffness of a user, and the like. Each setting may be iteratively updated based on data received from crowd-sourcing, user historical data, and the like. For instance, each setting may be continually updated to optimize a reduction of symptoms of most users from a population of a plurality of users. In some embodiments, a setting of the wearable device 100 may include an adaptive mode. An adaptive mode may include the waveform parameter selection 220 continually looking for a highest weight of sensor data 616 and/or most severe symptom and generating one or more waveform parameters to reduce said symptom and/or weight. An adaptive mode of the wearable device 100 may utilize a machine learning model, in some embodiments. An adaptive mode machine learning model may be trained with training data correlating sensor data and/or weights of sensor data to one or more waveform parameters. Training data may be received through user input, external computing devices, and/or previous iterations of processing. An adaptive mode machine learning model may be configured to input the filtered sensor data 216 and one or more optimal waveform parameters 220 to reduce a symptom having a highest severity. In some embodiments, an adaptive mode machine learning model may be trained remotely and weights of the trained model may be communicated to the wearable device 100 which may reduce processing load of the wearable device 100.

Referring now to FIG. 3, a block diagram of a waveform parameter selection process 300 through a mobile device is presented. The process 300 may be performed by a processor, such as processing unit 104, as described above with reference to FIG. 1, without limitation. The process 300 may include a waveform parameter selection 304. The waveform parameter selection 304 may be the same as the waveform parameter selection 220 as described above with reference to FIG. 6. In some embodiments, an application 308 may be configured to run on a computing device. The application 308 may be run on, but not limited to, laptops, desktops, tablets, smartphones, and the like. In some embodiments, the application 308 may take the form of a web application. The application 308 may be configured to display data to a user through a graphical user interface (GUI). A GUI may include one or more textual, pictorial, or other icons. A GUI generate by the application 308 may include one or more windows that may display data, such as images, text, and the like. A GUI generated by the application 308 may be configured to display sensor data, stimulation data, and the like. In some embodiments, a GUI generated by the application 308 may be configured to receiver user input 312. User input 312 may include, but is not limited to, keystrokes, mouse input, touch input, and the like. For instance, and without limitation, a user may click on an icon of a GUI generated by the application 708 that may trigger an event handler of the application 308 to perform one or more actions, such as, but not limited to, displaying data through a window, communicating data to another device, and the like. In some embodiments, user input 312 received through the application 708 may generate smartphone application data 316. The smartphone application data 316 may include one or more selections of one or more waveform parameters. In some embodiments, the smartphone application data 316 may include patient data such as, but not limited to, ages, onsets of one or more movement system disorders, comorbidities, activity level, body fat percentage, and/or other patient data. Patient data may be received through user input via application 308. In some embodiments, patient data may be received via one or more external computing devices and communicated to a processor running process 300 via Wi-Fi, Bluetooth, Cellular, or other signals. Waveform parameters may include, without limitation, amplitude frequency, and the like. In some embodiments, one or more waveform parameters may be generated based on patient data, such as, but not limited to, ages, onsets of one or more movement system disorders, comorbidities, activity levels of a patient, body fat percentages, and/or other patient data. Process 300 may automatically input patient data and select one or more optimal waveform parameters based on the patient data. Waveform parameters may be as described above with reference to FIGS. 1-2. As a non-limiting example, the smartphone application data 316 may include a selection of a higher frequency of a waveform output, the selection being generated by user input through the application 308.

Additionally, and/or alternatively, a user may generate user input 312 through one or more interactive elements of a wearable device. A wearable device may be as described above, without limitation, in FIG. 1. A wearable device may include one or more interactive elements such as, but not limited to, knobs, switches, buttons, sliders, and the like. Each interactive element of a wearable device may correspond to a function. For instance, a button of a wearable device may correspond to an increasing of a frequency of a waveform output while another button of the wearable device may correspond to a decreasing of a frequency of a waveform output. A user may generate device button data 320 through user input 312 of a wearable device. In some embodiments, a wearable device may include a touchscreen or other interactive display through which a user may generate device button data 320 from. In an embodiment, a wearable device may be configured to run the application 308 locally and receive the smartphone application data 316 through a touch screen or other input device that may be part of the wearable device. The waveform parameter selection 304 may be run locally on a wearable device and/or offloaded to one or more computing devices. In some embodiments, the waveform parameter selection 304 may be configured to receive the smartphone application data 316 and/or the device button data 320. The waveform parameter selection 304 may be configured to generate a waveform output, such as the stimulation output 128, based on the smartphone application data 316 and/or the device button data 320. A user may adjust the stimulation output 128 through generating the smartphone application data 316 and/or the device button data 320. The stimulation output 128 may be communicated to one or more parts of a peripheral nervous system 328 of a user, such as through one or more stimulators as described above with reference to FIG. 1.

Referring now to FIG. 4, a process 400 of feature extraction is presented. Process 400 may include inputting a filtered signal 424. Filtered signal 424 may be any filtered signal, such as those described above with reference to FIG. 3. Process 400 may extract temporal features, or one or more sets of temporal features. For instance, a first set of temporal features 404 may include, but us not limited to, minimum values, maximum values, first three standard deviation values, signal energy, root mean squared (RMS), and a zero crossing rate. A second set of temporal features 408 may include, but are not limited to, principal component analysis (PCA), kernel. A third set of temporal features 412 may include, but is not limited to, wavelet convolution, or autoconvolution. In some embodiments, each temporal feature may be extracted as a single set. Process 400 may extract spectral features 416. Examples of spectral features include the Fourier Transform, fundamental frequency, (Mel-frequency) Cepstral coefficients, the spectral centroid, and bandwidth. Features may be extracted with standard digital signal processing techniques onboard wearable device 100. A set of collected features may be fed into stimulation selection algorithm 420.

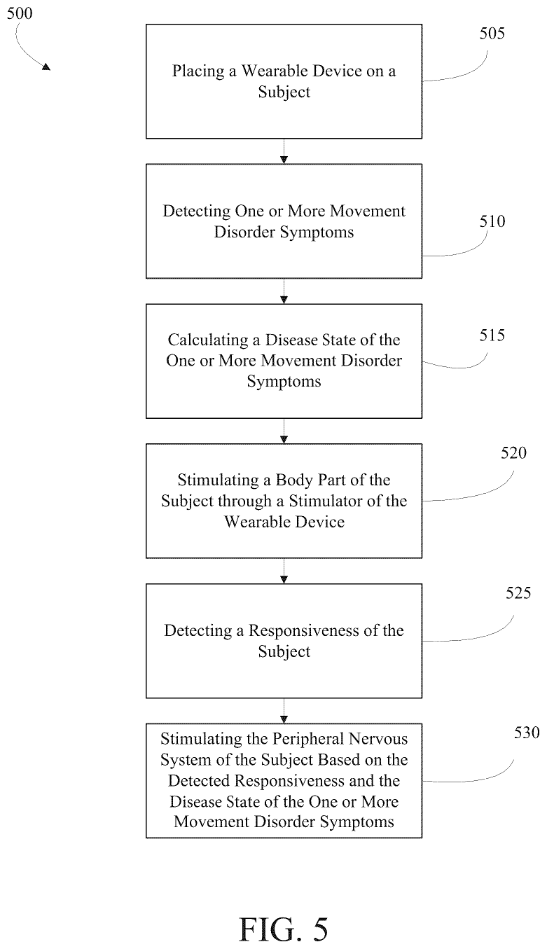

Referring now to FIG. 5, a method 500 of target peripheral stimulation is presented. At step 505, method 500 includes placing a wearable device on a user. A wearable device may include an attachment mechanism, such as, but not limited to, a wristband or straps. A wearable device may include one or more sensors, stimulators, and the like as described above with reference to FIG. 1.

At step 510, method 500 includes detecting one or more movement disorder symptoms. Detection of one or more movement disorder symptoms may occur through a sensor of a wearable device. For instance, a sensor may include an EKG, accelerometer, or other sensor, without limitation. One or more movement disorder symptoms may be detected based on changes in accelerometer values, in an embodiment. One or more movement disorder symptoms may include, but are not limited to, stiffness, rigidity, freezing, paralysis, paresis, dyskinesia, tremor, or a combination thereof. Detecting one or more movement disorder symptoms may include detecting an amplitude of a tremor. This step may be implemented as described above with reference to FIGS. 1-4, without limitation.

At step 515, method 500 includes calculating a disease state of the one or more movement disorder symptoms. A disease state may include, but is not limited to, a severity level, a stage of an onset, and/or a stage of one or more movement disorder symptoms. In some embodiments, calculating a disease state of the one or more movement disorder symptoms may include calculating each of a severity level, stage of an onset, and/or a stage of one or more movement disorder symptoms. In some embodiments, calculation of disease state of the one or more movement disorder symptoms may occur locally on a processor of a wearable device. In some embodiments, sensor data may be communicated to an external computing device via a communication module of a wearable device. An external computing device may calculate a disease state of the one or more movement disorder symptoms and may communicate the calculated disease state to a wearable device via a communication module of the wearable device. In some embodiments, calculation of disease state of the one or more movement disorder symptoms may include calculating a baseline UPDRS score of a user based on the detection of the one or more movement disorder symptoms. In some embodiments, calculation of disease state of the one or more movement disorder symptoms may include performing a quiet sitting and/or extended arm test. For instance, a baseline severity of a user may be calculated. A baseline severity may include an amplitude in tremor displacement of a body part of a user. A normal to slight baseline tremor may include about less than 1 cm in tremor amplitude. A mild to sever baseline tremor may include about 1 cm or greater than about 1 cm in tremor amplitude.

In some embodiments, calculating a disease state of the one or more movement disorder symptoms may include utilization of a machine learning model. For instance, a machine learning model may be trained with training data correlating one or more movement disorder symptoms to disease states of the one or more movement disorder symptoms. Training data may be received via user input, external computing devices, and/or previous iterations of processing. In some embodiments, parameters of a machine learning model trained to determine a disease states of the one or more movement disorder symptoms may be communicated to a processor of a wearable device via a communication module of the wearable device. A processor of a wearable device may utilize one or more machine learning parameters to perform one or more machine learning processes. In some embodiments, data may be communicated to an external computing device via a communication module of a wearable device. An external computing device may train and/or deploy one or more machine learning models to determine or predict a disease state of the one or more movement disorder symptoms. A determination or prediction may be sent to a processor of a wearable device from an external computing device via a communication module of the wearable device.

At step 520, method 500 includes stimulating a peripheral nervous system of the user through a stimulator of the wearable device. A stimulator may be vibratory, electric, ultrasonic, or other stimulator types. In some embodiments, a stimulator of a wearable device may be positioned to contact a surface of a body part of a user comprising peripheral nerve and/or tissue. For instance and without limitation, one or more stimulators of a wearable device may be positioned and/or embedded in a wrist band of the wearable device that, while placed on a user, contacts one or more areas of the user's wrist comprising peripheral nerve and/or tissue. In some embodiments, stimulation of a peripheral nervous system of a user may include providing a stimulation output to one or more body parts of a user comprising a peripheral nervous system. A stimulation output may be as described above with reference to FIG. 1. This step may be implemented, without limitation, as described above with reference to FIGS. 1-4.

At step 525, method 500 includes detecting a responsiveness of the user. A responsiveness may be detected via one or more sensors of a wearable device. A responsiveness may be calculated locally on a processor of a wearable device. In some embodiments, sensor data may be communicated to an external computing device via a communication module of a wearable device. An external computing device may calculate a responsiveness based on sensor data received by a wearable device. A responsiveness may be calculated by a change in a UPDRS score, root mean square of a tremor amplitude, quiet sitting test, and/or extended arm test. A responsiveness may be classified into various categories. For instance, a decrease in UPDRS score by 1 point or more may be classified as a high responsiveness. In some embodiments, a responsiveness of a user may be calculated by comparing a reduction in one or more movement disorder symptoms to an average reduction in one or more movement disorder symptoms across a cohort of similar individuals. For instance, average responsiveness may be calculated for certain age groups, such as individuals from about 18-25, individuals from about 26-35, individuals from about 36-50, and individuals above 50, without limitation. Average responsiveness may be generated for individuals of a certain physiology, such as body fat percentage, muscle mass, skeletal muscle mass, height, weight, and/or other features. For instance, those with more muscle mass may have a higher reduction in one or more movement disorder symptoms than those with less muscle mass. In some embodiments, various responsiveness and/or correlations of demographic data across a plurality of patients may be used in calculating one or more stimulation and/or waveform parameters of a stimulation output. Demographic data may include patient data as described above. For instance and without limitation, demographic data may include ages, muscle masses, ages of onset of one or more movement disorder symptoms, skeletal muscle mass, height, weight, and/or other features may be correlated to various levels of responsiveness, such as, but not limited to, low responsiveness, average responsiveness, high responsiveness, and/or other levels of responsiveness. Responsiveness and/or correlations of responsiveness to demographic data may be calculated locally and/or received via an external computing device. This step may be implemented without limitation as described above with reference to FIGS. 1-4.

At step 530, method 500 includes stimulating the peripheral nervous system of the user based on the detected responsiveness and the disease state of the one or more movement disorder symptoms. For instance, a stimulation output, such as described above with reference to FIG. 1, may be adjusted and/or tuned to account for a responsiveness and/or disease state of the one or more movement disorder symptoms. As a non-limiting example, a user classified as having a mild severity level and/or an early stage onset of one or more movement disorder symptoms may have a reduction in severity of the one or more movement disorder symptoms with a lower amplitude and/or frequency of a stimulation output than users classified as having a severe severity level and/or late stage onset of one or more movement disorder symptoms. In some embodiments, a machine learning model may be implemented to adjust one or more parameters of a simulation output based on a responsiveness, and/or disease state of one or more movement disorder symptoms of a user. A machine learning model may be trained with training data correlating responsiveness and/or disease states of one or more movement disorder symptoms with one or more stimulation parameters. Training data may be received via user input, external computing devices, and/or previous iterations of processing. A machine learning model may be trained to input sensor data, responsiveness and/or disease states of one or more movement disorder symptoms and output stimulation outputs and/or parameters thereof. A machine learning model may be trained externally from a wearable device, such as at an external computing device that may be in communication with a wearable device via a communication module of the wearable device. A wearable device may receive one or more outputs of a machine learning model from an external computing device, such as by a communication module of the wearable device, and may adjust one or more stimulation parameters of a stimulation output. In some embodiments, a wearable device may communicate suggested stimulation parameters to a user through a mobile application on a smartphone. Suggestions may include increasing or decreasing frequencies, pulse lengths, amplitudes, durations, and/or other parameters of a stimulation output.

In some embodiments, method 500 includes calculating a correlation between responsiveness and disease state of one or more movement disorder symptoms. Calculations of correlation may occur locally, such as on a processor of a wearable device. In some embodiments, a wearable device may communicate sensor data to an external computing device via a communication module of the wearable device. An external computing device may calculate correlations of responsiveness with one or more disease states of one or more movement disorder symptoms. In some embodiments, correlations between user physiology and/or responsiveness may be calculated. For instance and without limitation, users having a higher muscle mass may have an increase in reduction of one or more movement disorder symptoms while users having a lower muscle mass may have a lower increase in reduction of one or more movement disorder symptoms compared to the users with a higher muscle mass. Correlations may be calculated with any user data, such as, but not limited to, heights, weights, ages, muscle mass, body fat percentage, skeletal muscle mass, exercise regimes, bone density, and/or other user data. A machine learning model may be trained to input user data and output simulation outputs and/or parameters thereof based on training data correlating various user data to stimulation outputs and/or parameters thereof. Training data may be received via user input, external computing devices, and/or previous iterations of processing.

Referring now to FIG. 6, a graph showing baseline UPDRS scores of subjects is illustrated. In FIGS. 6-9, the tests were conducted on 17 tremor-dominant Parkinson's patients. The baseline demographics of the 17 patients were 59% male, 41% female, age ranges from 43-79 with an average of 66.9, age of an onset of movement symptom disorders of between 29-76 with an average age of onset of 59.6, and an average baseline UPDRS Tremor sub score of about 5.4 with ranges between 4-8.

Graphs 600A-B further shows percent improvement in root mean squared (RMS) tremor displacement in both a quiet sitting and extended arm test. Results are shown on a baseline Unified Parkinson's Disease Rating Scale (UPDRS), which comprises four parts: Part I evaluates mentation, behavior, and mood; Part II assesses activities of daily living; Part III evaluates motor skills; and Part IV assesses treatment-related motor and non-motor complications. Each part consists of various sub-items, all scored on a scale of 0-4, where 0 indicates no impairment and 4 signifies severe impairment. The extended arm test for postural tremor and the quiet sitting test for rest tremor are parts of the motor examination in Part III. Specifically, they are included in the evaluation of rigidity and postural stability.

The Tremor Research Group Essential Tremor Rating Assessment Scale (TETRAS) is broken down into four performance sections: Part A assesses tremor at rest; Part B evaluates kinetic tremor; Part C assesses postural tremor; and Part D evaluates the voice. Each of these parts contains a variety of sub-items, scored on a scale of 0-4, similar to the UPDRS. Furthermore, TETRAS includes a performance test (Part E), which tests writing and drawing abilities. The extended arm test is used in Part C for assessing postural tremor, while the quiet sitting test is used in Part A for assessing rest tremor. By looking at these sub-tests, clinicians can derive a comprehensive view of a patient's tremor severity, enabling them to effectively monitor changes and adjust treatment plans as necessary. Extended Arm Test for Postural Tremor—The extended arm test is a vital component of Part III of the UPDRS and Part C of TETRAS. This test specifically evaluates postural tremor, which manifests when a person maintains a position against gravity. During the test, the patient holds their arms extended in front of them, typically at shoulder height, for a certain period of time. The clinician then observes the frequency and amplitude of any tremors. This non-invasive and straightforward test enables the identification and assessment of conditions like essential tremor or Parkinson's disease, and can provide a measure of their severity or progression.

As part of Part III of the UPDRS and Part A of TETRAS, the quiet sitting test is used to measure rest tremor. This type of tremor appears when the muscles are relaxed and supported against gravity. In the test, the patient is asked to sit quietly with their hands resting on their laps. Any tremor's presence, frequency, and amplitude are then observed and rated. This easy-to-administer test is critical for diagnosing and monitoring Parkinson's disease, as rest tremor is a hallmark of this condition. By observing changes over time, clinicians can understand the disease's progression and adjust treatment as needed.

Scoring for both the extended arm test (postural tremor) and the quiet sitting test (rest tremor) in the UPDRS is done using a scale from 0 to 4. The scores are generally correlated with specific amplitude ranges observed during the tests. In the UPDRS, a score of 0 represents the absence of visible tremor (0 cm amplitude). A score of 1 represents a slight tremor with an amplitude of less than 1 cm. A score of 2 indicates a mild but visible tremor with an amplitude ranging from 1 cm to 3 cm. A score of 3 denotes a moderate tremor with amplitudes between 3 cm and 10 cm, typically interfering with daily activities. Finally, a score of 4 is given for severe tremors with an amplitude greater than 10 cm, significantly impeding the execution of daily tasks.

As in the UPDRS, scoring for both the extended arm test (postural tremor) and the quiet sitting test (rest tremor) in the TETRAS scales is typically done using a scale from 0 to 4. In the TETRAS, a score of 0 represents the absence of visible tremor (0 cm amplitude). A score of 1 represents a barely visible tremor. A score of 1.5 represents a slight tremor with an amplitude of less than 1 cm. A score of 2 indicates a mild but visible tremor with an amplitude ranging from 1 cm to 3 cm. A score of 2.5 is given for a tremor that is between 3 cm and 5 cm. A score of 3 denotes a moderate tremor with amplitudes between 5 cm and 10 cm, typically interfering with daily activities. A score of 3.5 indicates a tremor ranging from 10 cm to 20 cm. Finally, a score of 4 is given for severe tremors with an amplitude greater than 20 cm, significantly impeding the execution of daily tasks. These scores offer an objective and quantifiable measure of tremor severity, allowing for monitoring of disease progression and treatment efficacy.

Referring still to FIG. 6, graph 600A shows baseline UPDRS scores for a quiet sitting test. Graph 600B shows baseline UPDRS scores for an extended arm test. In graph 600A, patients with a baseline UPDRS score of 0.0 had about a 0% improvement in RMS tremor displacement. Patients with a 1.0 baseline UPDRS score had about a 0% to −280% improvement in RMS tremor displacement. Patients with a 2.0 baseline UPDRS score had about a 10% improvement in RMS tremor displacement. Patients with a baseline UPDRS score of about 3.0 had about a 20% improvement in RMS tremor displacement.

Referring to graph 600B, results of an extended arm test are presented. Patients with a 0.0 baseline UPDRS score had about a 0% improvement in RMS tremor displacement. Patients with a 1.0 baseline UPDRS score had about a 0% improvement in RMS tremor displacement. Patients with a 2.0 UPDRS score had about a 5% to about 10% improvement in RMS tremor displacement. Patients with a 3.0 UPDRS score had about a 20% to 30% improvement in RMS tremor displacement.

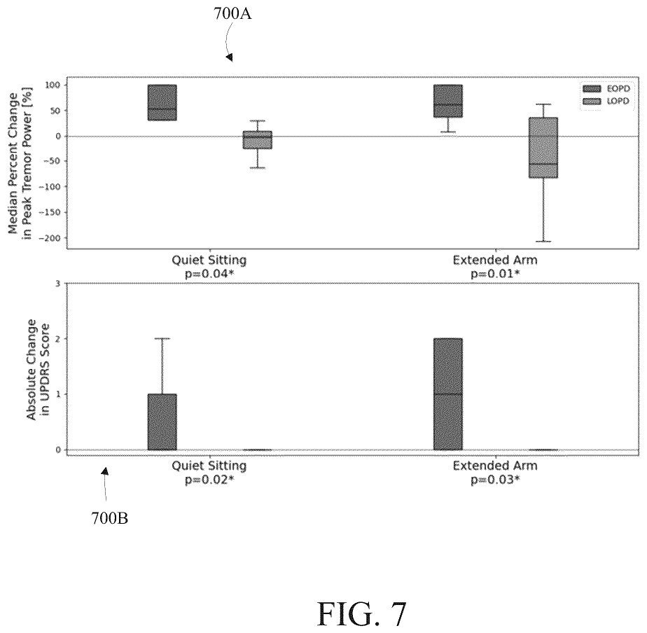

Referring now to FIG. 7, graphs showing median percent change and absolute change in symptoms of early and late onset Parkinson's disorder subjects is illustrated. In particular, results in a quiet sitting and extended arm test of early onset Parkinson's disorder (EOPD) and late onset Parkinson's disorder (LOPD) is presented. Graph 700A shows median percent change in peak tremor power for both EOPD and LOPD patients. Graph 700B shows the absolute change in UPDRS score in both the quiet sitting and extended arm tests for EOPD patients. Referring to graph 700A, EOPD patients had a median percent change in peak tremor power of about 40% in a quiet sitting test and about 50% in an extended arm test. LOPD patients had about a 0% median percent change in peak tremor power in a quiet sitting test and about a −100% median percent change in peak tremor power in an extended arm test.

Referring to graph 700B, EOPD patients had an absolute change in UPDS score of about 1 in a quiet sitting test and about 2 in an extended arm test while LOPD patients had no absolute change in UPDRS score.

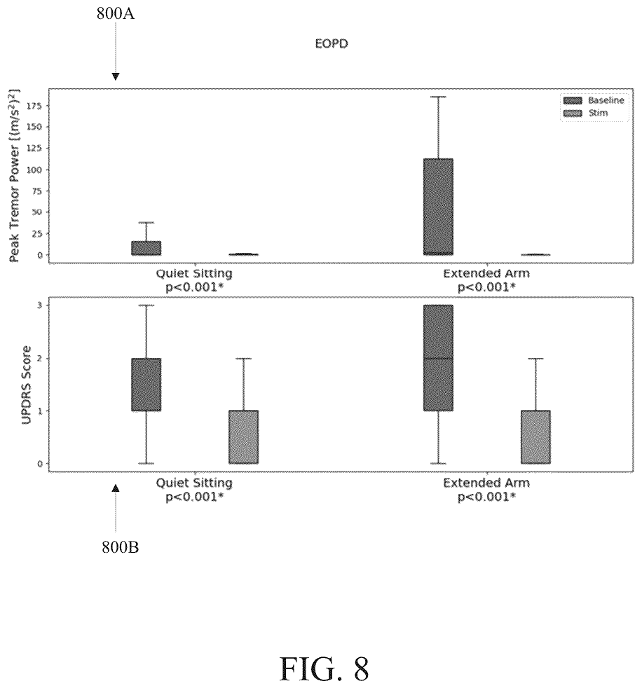

Referring now to FIG. 8, a graph showing results of baseline and stimulated EOPD subjects is presented. In particular, graphs 800A-B results of a quiet sitting and extended arm test for EOPD patients is presented. Graph 800A illustrates baseline scores of EOPD patients in both the quiet sitting and extended arm tests. Graph 800B illustrates results in UPDRS scores of the EOPD patients with stimulation active compared to non-stimulated EOPD patients.

Referring to graph 800A, EOPD patients had a baseline in peak tremor power of about 10 (m/s²)² in a quiet sitting test and a peak tremor power of about 125 (m/s²)² in an extended armor test. Stimulated EOPD patients had no discernable change in peak tremor power. Referring to graph 800B, EOPD patients had a baseline UPDRS score of about 1 to about 2 in a quiet sitting test. EOPD patients that were stimulated had a baseline UPDRS score of about 0 to about 1 in a quiet sitting test. In an extended arm test, EOPD patients had a baseline UPDRS score of about 1 to about 3 while stimulated EOPD patients had a UPDRS score of about 0 to about 1.

Referring now to FIG. 9, a graph showing results of baseline and stimulated LOPD subjects is presented. In particular, graphs 900A-B show results of a quiet sitting and extended arm test of LOPD patients with and without stimulation is presented. Graph 900A illustrates peak tremor power and graph 900B represents UPDRS scores. Referring to graph 900A, LOPD patients had a baseline peak tremor power of about 0 to about 5 (m/s²)² in a quiet sitting test and a baseline peak tremor power of about 14 (m/s²)² in an extended arm test. Stimulated LOPD patients had a peak tremor power of about 0 to about 7.5 (m/s²)² in a quiet sitting test. In an extended arm test, LOPD patients had a baseline UPDRS score of about 1 to about 2 and stimulated LOPD patients had no discernible difference.

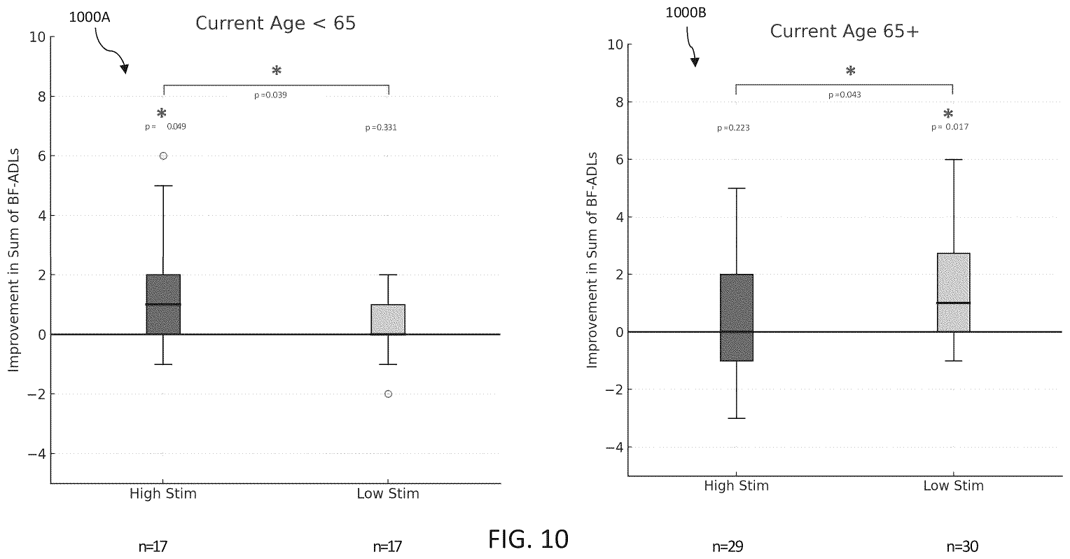

Referring now to FIG. 10, a graphs showing an improvement in BF-ADL scores in subjects of various ages is presented. Different stimulation parameters depending on subject age were used. This data is extracted from a clinical trial involving 47 subjects with definite or probable essential tremor, as defined by the Tremor Investigation Group (TRIG) criteria. 46% of subjects were male and 54% were female. The average age at time of testing was 65.71±9.61 years. Graph 1000A shows the results from each “high” and “low” levels of stimulation in a cohort of subjects who were younger than 65 years old at time of testing. Graph 1000B shows the results in a cohort of subjects who were at least 65 years of age at time of testing. The “high” stimulation level performed significantly better than the “low” level in the younger cohort (p=0.039), and vice versa in the older cohort (p=0.043). These results suggest the possibility for automated stimulation adjustment based on demographic characteristics.

The Bain and Findley Activities of Daily Living (BF-ADLs) are often used to measure patient-reported disability. The BF-ADL scale is one such measure, and it involves a survey-based assessment of a subject's disability as it relates to certain common tasks, such as eating soup or dialing a telephone. This scale has been employed in real-time, wherein a subject is asked to complete a task and determine whether they were 1-able to do the task without difficulty, 2-able to do the task with a little effort, 3-able to do the task with a lot of effort, or 4-unable to do the task by yourself. The scale is aimed to measure a subject's own assessment of their disability, and was developed to evaluate the severity of tremor in patients with essential tremor and postural limb tremor associated with dystonia.

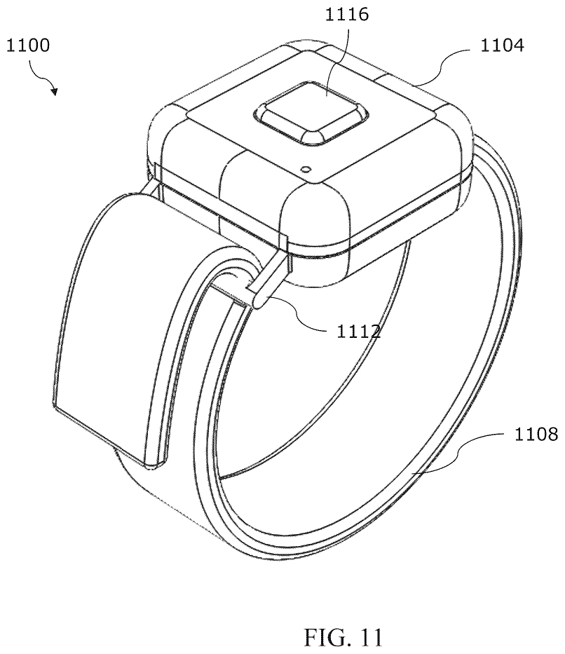

Referring now to FIG. 11, an illustration of a wearable device 1100 is presented. In some embodiments, the wearable device 1100 may include a housing 1104 that may be configured to house one or more components of the wearable device 1100. For instance, the housing 1104 of the wearable device 1100 may include a circular, ovular, rectangular, square, or other shaped material. In some embodiments, the housing 1104 may have a length of about 5 inches, a length of about 5 inches, and a width of about 5 inches, without limitation. In some embodiments, the housing 1104 may have a length of about 1.5 inches, a width of about 1.5 inches and a height of about 0.5 inches. The housing 1104 of the wearable device 1100 may have an interior and an exterior. An interior of the housing 1104 of the wearable device 1100 may include, but is not limited to, one or more sensors, stimulators, energy sources, processors, memories, and the like, such as those described above with reference to FIG. 1. In some embodiments, an exterior of the housing 1104 of the wearable device 1100 may include one or more interactive elements 1116. An “interactive element” as used in this disclosure is a component that is configured to be responsive to user input. The interactive element 1116 may include, but is not limited to, buttons, switches, and the like. In some embodiments the wearable device 1100 may have a singular interactive element 1116. In other embodiments, the wearable device 1100 may have two or more interactive elements 1116. In embodiments where the wearable device 1100 has a plurality of interactive elements 1116, each interactive element 1116 may correspond to a different function. For instance, a first interactive element 1116 may correspond to a power function, a second interactive element 1116 may correspond to a waveform adjustment, a third interactive element 1116 may correspond to a mode of the wearable device 1100, and the like. In some embodiments, the wearable device 1100 may include a touch screen display.

In some embodiments, the wearable device 1100 may include one or more batteries. For instance, and without limitation, the wearable device 1100 may include one or more replaceable batteries, such as lead-acid, nickel-cadmium, nickel-metal hydride, lithium-ion, and/or other battery types. The housing 1104 of the wearable device 1100 may include a charging port that may allow access to a rechargeable battery of the wearable device 1100. For instance and without limitation, the wearable device 1100 may include one or more rechargeable lithium-ion battery and a charging port of the housing 1104 of the wearable device 1100 may be a USB-C, micro-USB, and/or other type of port. A battery of the wearable device 1100 may be configured to charge at a rate of about 10 W/hr. A battery of the wearable device 1100 may be configured to charge at about 3.7V with a current draw of about 630 mA. A battery of the wearable device 1100 may have a capacity of about 2.5 Wh, greater than 2.5 Wh, or less than 2.5 Wh, without limitation. In some embodiments, the wearable device 1100 may include one or more wireless charging circuits that may be configured to receive power via electromagnetic waves. The wearable device 1100 may be configured to be charged wirelessly at a rate of about 5 W/hr through a charging pad or other wireless power transmission system. In some embodiments, a battery of the wearable device 1100 may be configured to be charged at about 460 mA, greater than 460 mA, or less than 460 mA.

Still referring to FIG. 11, the wearable device 1100 may include an attachment system. An attachment system may include any component configured to secure two or more elements together. For instance, and without limitation, the wearable device 1100 may include a wristband 1108. The wristband 1108 may include one or more layers of a material. For instance and without limitation, the wristband 1108 may include multiple layers of a polymer, such as rubber. The wristband 1108 may have an interior and an exterior. An interior and an exterior of the wristband 1108 may be a same material, texture, and the like. In other embodiments, an interior of the wristband 1108 may be softer and/or smoother than an exterior of the wristband 1108. As a non-limiting example, an interior of the wristband 1108 may be a smooth rubber material while an exterior of the wristband 1108 may be a Velcro material. The wristband 1108 may have a thickness of about 2 mm. In other embodiments, the wristband 1108 may have a thickness of greater than or less than about 2 mm. The wristband 1108 may be a rubber band, Velcro strap, and the like. In some embodiments, the wristband 1108 may be adjustable. For instance, the wristband 1108 may be a flexible loop that may self-attach through a Velcro attachment system. In some embodiments, the wristband 1108 may attach to one or more hooks 1112 of an exterior of the housing 1104 of the wearable device 1100. In some embodiments, the wristband 1108 may be magnetic. In other embodiments, the wristband 1108 may include a column, grid, or other arrangement of holes that may receive a latching from the hook 1112.

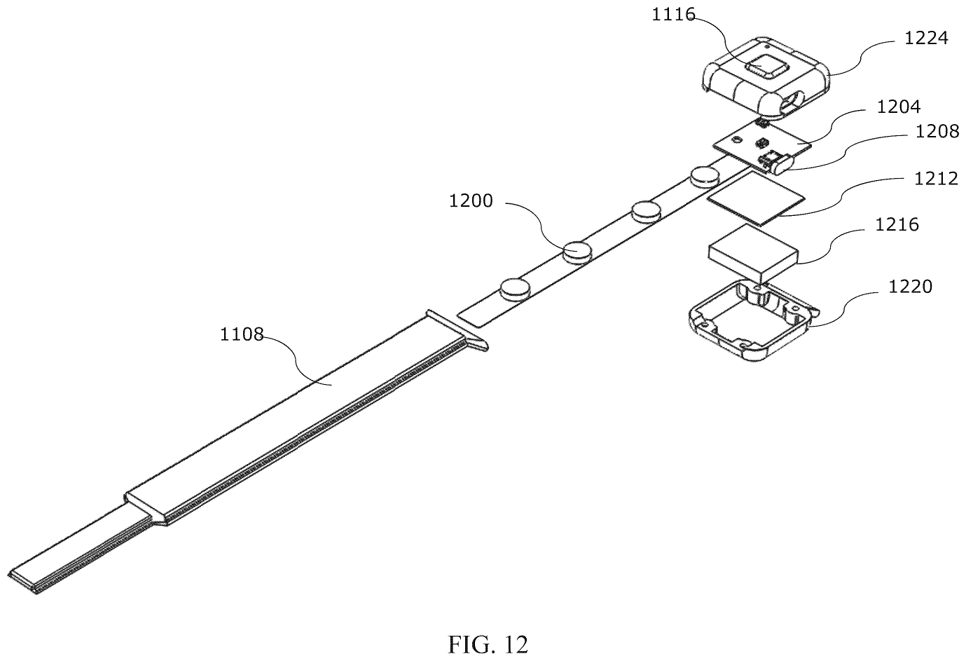

Referring now to FIG. 12, an exploded side view of the wearable device 1100 is shown. The wearable device 1100 may include stimulators 1200. The mechanical transducers 1200 may be housed within the wristband 1208. The wristband 1208 may be configured to interface with a user's writs. The wearable device 1100 may have a top half of a housing 1224 and a bottom half of a housing 1220. In some embodiments, between the top half 1224 and the bottom half 1220, a printed circuit board 1204 (PCB) may be positioned. Further, a silicone square may be positioned to insulate a bottom of the PCB 43, which may be positioned above a battery 1216. The battery 1216 may include protection circuitry to protect from overcharging and unwanted discharging. In some embodiments, the wearable device 1000 may include a magnetic connector 1208. The magnetic connector 1208 may be configured to align the wearable device 1100 with a charging pad, station, and the like. The magnetic connector 1208 may be configured to receive power wirelessly to recharge the battery 1216. The magnetic connector 1208 may be coupled to the battery 1216 and mounted in the housing 1220 and/or 1224. In some embodiments, the magnetic connector 1208 may be inserted into the PCB 1204. The magnetic connector 1208 may be configured to mate with a connector from an external charger.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A wearable device for targeted peripheral stimulation, comprising:
a processor;
a memory communicatively connected to the processor, the memory containing instructions configuring the processor to:
determine a disease state of one or more movement disorder symptoms of a user;
generate a stimulation output based on the one or more movement disorder symptoms and the disease state of the movement disorder symptoms; and
command a stimulator in communication with the processor to apply the stimulation output to a peripheral nervous system of the user to reduce the one or more movement disorder symptoms.

2. The wearable device of claim 1, further comprising a sensor in communication with the processor, the sensor configured to output sensor data indicative of the one or more movement symptoms disorders of the user, wherein the processor is further configured to:
calculate a level of responsiveness of the user based on the sensor data generated while the stimulation output is applied; and
communicate the responsiveness to an external computing device via a wireless communication unit of the wearable device.

3. The wearable device of claim 4, wherein the level of responsiveness of the user is calculated as one of low responsiveness, average responsiveness, or high responsiveness.

4. The wearable device of claim 1, wherein the reduction in the one or more movement disorder symptoms is greater in users with a disease state comprising an early stage of onset of one or more movement disorder symptoms than users with a disease state comprising a late stage of onset of one or more movement disorder symptoms.

5. The wearable device of claim 1, wherein the responsiveness is influenced at least in part by demographic data of the user.

6. The wearable device of claim 1, wherein the one or more movement disorder symptoms are one of tremor, stiffness, rigidity, freezing, paralysis, paresis, dyskinesia, or a combination thereof.

7. The wearable device of claim 1, wherein the stimulation output is applied to a proprioceptive nerve or proprioceptive tissue of one of flexor carpi radialis, flexor carpi ulnaris, extensor carpi radialis, extensor carpi ulnaris, or a combination thereof of the peripheral nervous system of the user.

8. The wearable device of claim 1, wherein the processor is further configured to:
adjust the stimulation output based on the disease state of the movement disorder symptoms; and
command the one or more stimulators to apply the adjusted stimulation output to the peripheral nervous system of the user.

9. The wearable device of claim 1, wherein the processor is further configured to generate the stimulation output through a stimulation selection algorithm, wherein the stimulation selection algorithm generates the stimulation output at least in part on patient data communicated to the processor.

10. The wearable device of claim 9, wherein the disease state comprises one of a severity level of the one or more movement disorder symptoms, stage of onset of the one or more movement symptoms, stage of movement disorder of the one or more movement disorder symptoms, or combination thereof.

11. A method of target peripheral stimulation, comprising:
placing a wearable device on a user;
detecting one or more movement disorder symptoms of the user through a sensor of the wearable device;
calculating a disease state of the one or more movement disorder symptoms based on the detection;
stimulating a peripheral nervous system of the user through a stimulator of the wearable device in response to the one or more movement disorder symptoms;
detecting a responsiveness of the user through the sensor of the wearable device; and
stimulating the peripheral nervous system of the user based on the detected responsiveness and the stage of the disease state of the one or more movement disorder symptoms.

12. The method of claim 11, wherein the disease state comprises one of a severity level of the one or more movement disorder symptoms, stage of onset of the one or more movement symptoms, stage of movement disorder of the one or more movement disorder symptoms, or combination thereof.

13. The method of claim 11, further comprising calculating a correlation between the responsiveness of the user and the disease state of the one or more movement disorder symptoms.

14. The method of claim 13, further comprising adjusting, based on the correlation, one or more parameters of a stimulation output of the wearable device.

15. The method of claim 13, wherein an early stage of an onset of the disease state is correlated to a high responsiveness to the stimulation.

16. The method of claim 13, wherein a mild or late stage of an onset of the one or more movement disorder symptoms of the disease state is correlated to a low responsiveness to the stimulation.

17. The method of claim 11, wherein a peripheral nerve or tissue in a wrist or arm of the user is stimulated.

18. The method of claim 11, wherein stimulating the body part further comprises providing one or more of vibratory, electrical, or ultrasonic output or a combination thereof to the peripheral nervous system through the stimulator of the wearable device.

19. The method of claim 11, wherein the reduction in the one or more movement disorder symptoms is greater in users with a mild severity level than users with a moderate or sever severity level of one or more movement disorder symptoms.

20. The method of claim 11, wherein the responsiveness is influenced at least in part patient data of the user.
